(19) United States
(12) Patent Application Publication
(10) Pub. No.: US 2013/0253412 A1
(43) Pub. Date: Sep. 26, 2013

(54) ROLLER-BALL APPLICATOR FOR TRANSDERMAL IONTOPHORETIC DELIVERY

(71) Applicants: Jamal Yanaki, Salt Lake City, UT (US); Ralph Koschinsky, Sandy, UT (US)

(72) Inventors: Jamal Yanaki, Salt Lake City, UT (US); Ralph Koschinsky, Sandy, UT (US)

(73) Assignee: ACTIVATEK, INC.

(21) Appl. No.: 13/430,313

(22) Filed: Mar. 26, 2012

(51) Int. Cl. A61N 1/30

(52) U.S. Cl. 604/20; 604/503

(57) ABSTRACT

Embodiments of iontophoretic devices are disclosed which may include a reservoir; a roller-ball applicator coupled to the reservoir and configured to dispense contents of the reservoir; a power supply; a first electrode electrically coupled to a pole of the power supply; and a second electrode coupled to the other pole of the power supply. The devices may be configured such that a current travels between the first electrode and the second electrode through the skin of a subject when the device is used.

### FIELD

This application relates to the transdermal administration of ionic solutions, drugs and cosmetics.

More particularly, this application relates to active iontophoretic delivery systems in which a hand-held roller-ball applicator forming an electrical contact applied to the surface of the skin of a subject for the purpose of delivering agents through the surface of the skin into underlying tissues.

### BACKGROUND

Iontophoresis uses electrical current through a body, such as human or animal, to drive charged ionic particles into the skin. Some ionic particles may be drugs or cosmetic solution. During active iontophoresis, direct electrical current is used to cause ions of a solution, such as a medicament, to move across the surface of the skin and to diffuse into underlying tissue. The surface of the skin is not broken by the iontophoresis. When conducted within appropriate parameters, the sensations experienced by a subject during the delivery of the ionic solution in this manner are not unpleasant. Therefore, active iontophoresis presents an attractive alternative to hypodermic injections and to intravascular catheterization.

Iontophoresis has been used with patches that allow a current flow and ionic treatment of a particular area of the body. As such, conventional iontophoresis has used patches to allow sufficient time for the current to drive an ionic solution into a particular part of the body. Due to the limitations of current and patch size, only specific, discrete areas of skin covered by an iontophoretic patch accept the transdermal ionic solution transfer.

### SUMMARY

Embodiments of iontophoretic devices are disclosed which may include a reservoir; a roller-ball applicator coupled to the reservoir and configured to dispense contents of the reservoir; a power supply; a first electrode electrically coupled to a pole of the power supply; and a second electrode coupled to the other pole of the power supply. The devices may be configured such that a current travels between the first electrode and the second electrode through the skin of a subject when the device is used.

In some embodiments, the roller-ball applicator may include the first electrode, a roller-ball, and a fitment, the roller-ball comprising a conductive material. In such embodiments, the roller-ball may function as the first electrode. Similarly, some embodiments may include a handle, wherein the second electrode is included on the handle, such that the second electrode may be configured to be grasped by the hand of the subject. The reservoir may contain an ionic solution. Iontophoretic devices may also include a control circuit disposed between the power supply and one of the first and second electrodes. The device may be configured for a single use only, or the reservoir may be refillable or replaceable. The device may also include a cap to fit over the roller-ball applicator.

Some exemplary methods of iontophoretic application may include placing ionic fluid in a reservoir; coupling the reservoir to a roller-ball applicator, the roller-ball applicator having a roller-ball and a handle; grasping the handle; placing the roller-ball against the skin of an individual; dispensing the ionic fluid by way of the roller-ball onto the skin of the individual; and applying current between the roller-ball and the skin of the individual such that the ionic fluid is delivered iontophoretic ally into the skin of the individual.

### BRIEF DESCRIPTION OF THE DRAWINGS

The following description can be better understood in light of Figures, in which:

FIG. 1 illustrates a schematic view of an exemplary iontophoretic roller-ball applicator;

FIG. 1b illustrates schematic view of an exemplary iontophoretic roller-ball applicator being used;

FIGS. 2a and 2b illustrate an exemplary iontophoretic roller-ball applicator;

FIG. 3 illustrates a cross-sectional view of the exemplary iontophoretic roller-ball applicator of FIG. 2a;

FIG. 4 illustrates an exploded view of the exemplary roller-ball applicator of FIG. 3;

FIGS. 5 and 6 illustrate exemplary embodiments of portions of iontophoretic roller-ball applicators;

FIG. 7 illustrates an exemplary iontophoretic roller-ball applicator; and

FIG. 8 illustrates an exemplary iontophoretic roller-ball applicator.

Together with the following description, the Figures demonstrate and explain the principles of iontophoretic roller-ball applicators and methods for making and using the iontophoretic roller-ball applicator. In the Figures, the thickness and configuration of components may be exaggerated for clarity. The same reference numerals in different Figures represent the same component.

### DETAILED DESCRIPTION

The following description supplies specific details in order to provide a thorough understanding. Nevertheless, the skilled artisan would understand that the apparatus and associated methods of using the apparatus can be implemented and used without employing these specific details. Indeed, the apparatus and associated methods can be placed into practice by modifying the illustrated apparatus and associated methods and can be used in conjunction with any other apparatus and techniques conventionally used in the industry. For example, while the description below focuses on iontophoretic roller-ball applicators powered with a battery, applicators may also be powered from a wall socket.

Iontophoretic roller-ball applicators described below may provide a user with a manipulable device that may used to apply ionic solution, such as a medicament, to treat an area of skin with iontophoresis. Iontophoretic roller-ball applicators may provide additional massaging or stimulation to skin to facilitate the iontophoresis and to provide the iontophoresis to a larger area of skin than traditional attached patches, which are placed and held in a single location. Additionally, the ionic solution applied to the skin may be conductive and continue with iontophoresis over a broad area when in conductive contact with the roller-ball of an iontophoretic roller-ball applicator, which may function as an electrode in the iontophoresis process, as will be explained in more detail below.

The direct current employed in active iontophoresis systems may be obtained from a variety of electrical power sources. These may include electrical equipment that ultimately receives power from a wall socket, paired regions of contrasting galvanic materials that when coupled by a fluid or a gel medium produce minute electrical currents, capacitors, consumable and rechargeable batteries, etc. A flow of electrical current may require an uninterrupted, electrically-conductive pathway from the positive pole of a power source to the other, negative pole of the power source. Living tissue may be made up primarily of fluid and may be, therefore, a conductor of electrical current. In an iontophoretic circuit, the opposite poles of a power source may be electrically coupled to respective, separated contact locations on the skin of the subject. The difference in electrical potential created by the power source between those contact locations may cause a movement of electrons and electrically charged molecules, or ions, through the tissue between the contact locations.

In an active iontophoretic delivery system, the polarity of the net overall electrical charge on dissolved molecules of an ionic solution, including solutions with medicaments and/or cosmetics, may determine the contact location on the skin at which a supply of the ionic solution of must be positioned. A positively charged ionic solution in a reservoir against the skin of a patient may be coupled to the positive pole of any power source that is to be used to administer the ionic solution iontophoretically. Correspondingly, a reservoir on the skin of a patient containing a negatively charged ionic solution may be coupled to the negative pole of such a power source. Examples of common iontophoretically administrable ionic solutions of positive polarity include Bupivacaine hydrochloride, Calcium chloride, Lidocaine hydrochloride, Zinc chloride, and Lidocaine. Examples of common iontophoretically administrable ionic solutions of negative polarity include Betamethasone sodium phosphate, Dexamethasone sodium phosphate, Fentinol, Copper sulfate, Acetic acid, Magnesium sulfate, Naproxen sodium, Sodium chloride, and Sodium salicylate.

The ionic solution supply may be housed in a fluid reservoir that is positioned electrically conductively engaging the skin of the subject at an anatomical location overlying the tissue to which ionic solution is to be administered. The ionic solution reservoir can take the form of a gel suspension of the ionic solution or of a pad of an absorbent matrix, such as gauze or cotton, which is saturated with fluid containing the ionic solution. In some instances the fluid containing the ionic solution is provided from the manufacturer in the absorbent matrix. More commonly, the fluid is added to the absorbent matrix by a medical practitioner at the time that the ionic solution is about to be administered to a subject.

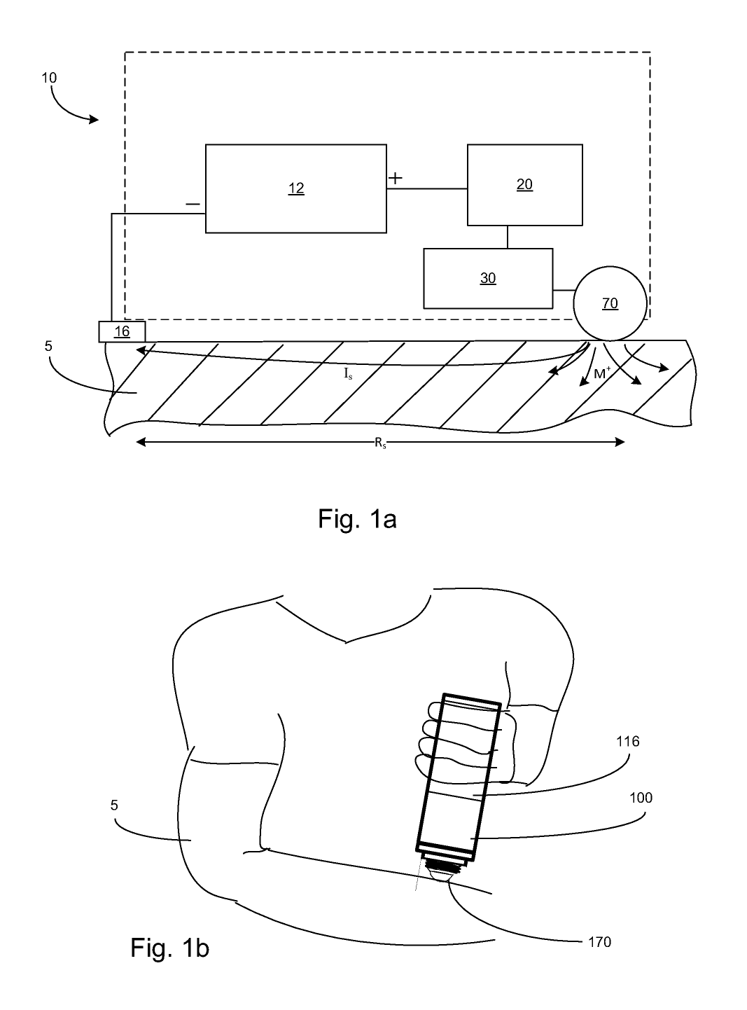

As shown in FIG. 1a, an iontophoretic circuit 10 for driving an ionic solution through unbroken skin 5 may be established by coupling the appropriate pole of power source 12 through ionic solution reservoir 30 and through roller-ball 70 to skin 5 of a subject at the anatomical location at which the ionic solution is to be administered. Roller-ball 70 may function as an electrode connected to one pole of power source 12 and provide an electrical pathway from power source 12 to skin 5 of a subject. Simultaneously, the other electrode 16 may be coupled to a pole of power source 12 may be coupled to an anatomical location on skin 5 of the subject that is distanced from ionic solution reservoir 30 and roller-ball 70. For example, a negative pole may be connected to electrode 116, which may be contacted by the hand of an individual holding an iontophoretic applicator 100 while roller-ball 170 applies ionic solution at a desired location, as shown in FIG. 1b.

The coupling of each pole of power source 12 may be affected by the electrical connection of each pole to a respective electrode. The electrode forming the roller-ball and delivering the ionic solution from reservoir 30 may be referred to as an active electrode; the electrode 16 at the location on skin 5 distanced from the roller-ball 70 may be referred to as a return electrode. The electrical potential that is imposed across ionic solution reservoir 30 of an iontophoretic circuit may produce electrical current flow Iₛ by causing electrolysis in some of the molecules of the water (H₂O) in the solution in reservoir 30. Skin 5 provides resistance Rₛ for circuit 10 between electrode 16 and roller-ball 70. Control circuit 20 may regulate and control current through circuit 10.

In some embodiments, such as when a person is using the applicator 100 to treat another person, a separate contact (not shown) between the skin 5 of the individual being treated and the other person performing the treatment may be provided to close the circuit between the electrode 16 and the skin 5 of the individual being treated. In some embodiments, this may simply be the person performing the treatment touching in a skin-to-skin manner the person being treated, or a cord extending from the applicator 100 with a remote electrode may be placed on or attached to the skin 5 of the individual being treated.

In electrolysis, the positively-charged hydrogen ion (H³⁰ ) of a water molecule may then becomes separated from the negatively-charged hydroxyl radical (HO⁻) of that same molecule. These ions and radicals may then migrate in respective opposite directions through the solution in the ionic solution reservoir. The hydrogen ions (H⁺) may then move toward the negative pole of the electrical potential being imposed on the solution, while the hydroxyl radicals (HO⁻) move toward the positive pole. This may in turn drive the treatment solution ions M⁺ into skin 5.

Ionic solution reservoir 30 may be contained within a roller-ball applicator, with an associated active electrode conveniently retained against skin 5 by contact with roller-ball 70, with roller-ball 70 functioning as the electrode as described in detail below, while return electrode 16 may be retained against skin 5 through the handle of the roller-ball applicator.

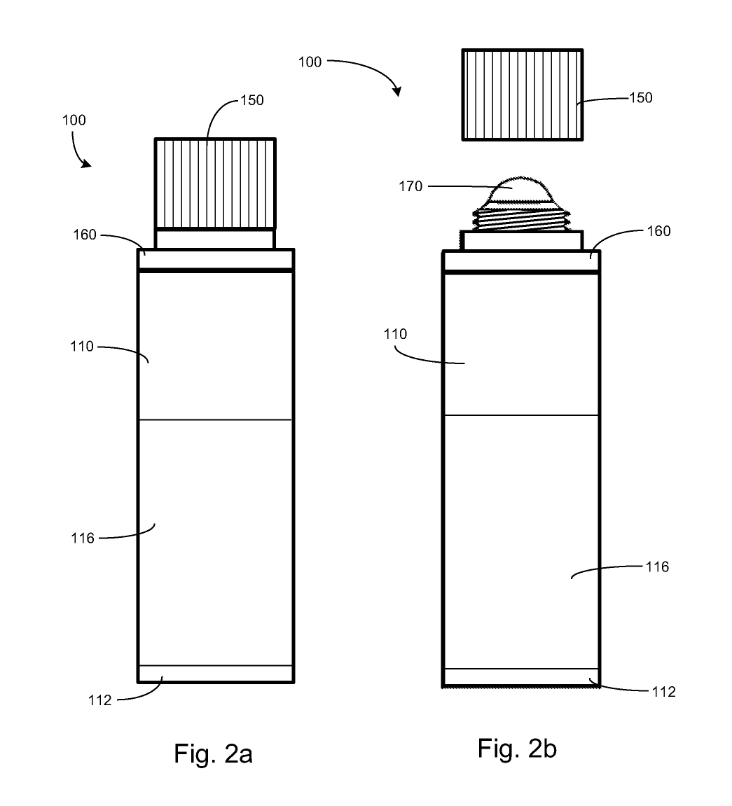

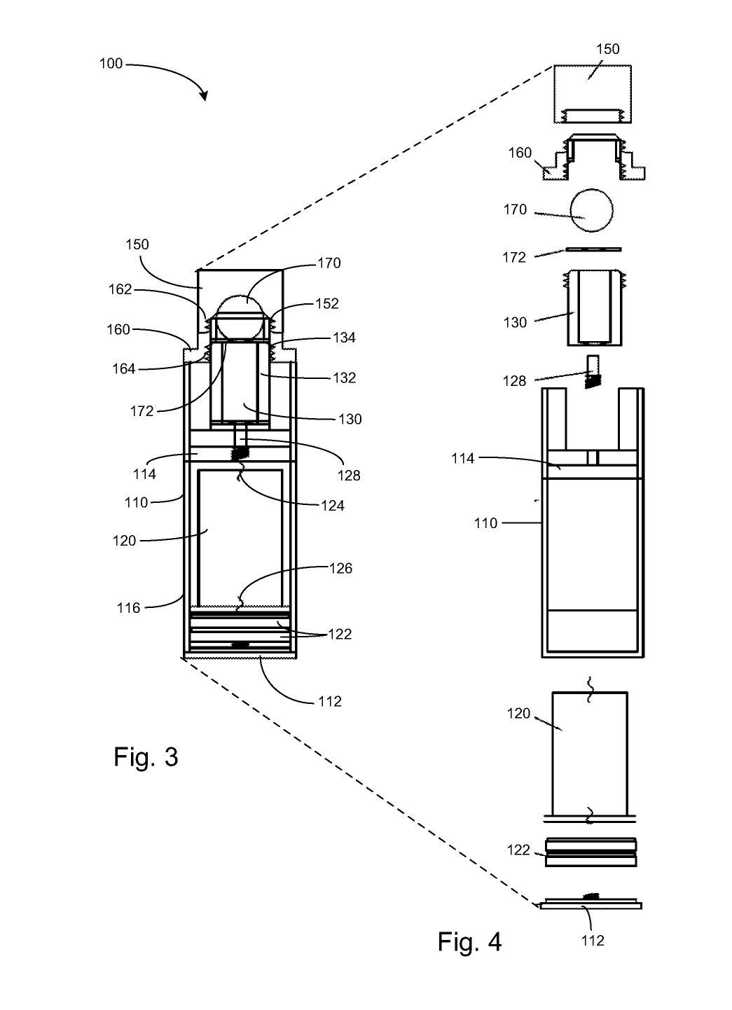

FIGS. 2a and 2b illustrate an embodiment of iontophoretic roller-ball applicator 100 Iontophoretic roller-ball applicator 100 may include body 110 with electrode 116, base 112, solution fitment 160 with roller-ball 170, and cap 150. FIGS. 3 and 4 illustrate the components of some embodiments of iontophoretic roller-ball applicator 100 in greater detail. In some embodiments, iontophoretic roller-ball applicators described may be reusable, or may be a single use applicator.

In the embodiments of FIGS. 3 and 4, body 110 houses various components for iontophoretic roller-ball applicator 100. Base 112 may be removably or permanently connected to body 110. Base 112 may also provide an electrical pathway between power source 122 and electrode 116, located on the outer surface of body 110. Body 110 may be formed such that it may be grasped by the hand of an individual, with electrode 116 being located to contact the hand of the individual grasping body 110. Body 110 may be formed of a non-conductive material to provide insulation between electrode 116 and roller-ball 170.

Base 112 may be formed of any suitable material, such as a conductive material, or a non-conductive material with a conductor to connect power source 122 to electrode 116. Some suitable materials may include metals, a plastic with a conductive coating, conductive plastic, conductive ceramic, conductive carbon etc. Power source 122 may be any suitable power source such as batteries, which may also include board circuitry to control current output, a connection to a wall outlet, capacitors, etc., to provide sufficient current over time to affect an iontophoretic process, for example between about 0.00001-100 mAmps. Power source 122 may be DC to provide a steady current to drive an ionic solution into the skin of a subject using iontophoretic roller-ball applicator 100. Lead 126 may connect power source 122 to control circuit 120. Control circuit 120 may function to provide and maintain the correct current flow between electrode 116 and roller-ball 170 when being used.

Lead 124 may connect control circuit 120 to conductive post 128 through plate 114. Plate 114 may function to separate and protect the electronic components in the lower portion of body 110 from the upper portion of body 110, which houses reservoir 130 of ionic solution. Plate 114 may be formed of a non-conductive material, such as plastic, or any other suitable material. In some embodiments, control circuit 120 may be directly connected to power source 122 external to base 112.

In the embodiment of FIGS. 3 and 4, conductive post 128 may contact reservoir 130. Reservoir 130 may be formed of a conductive material that allows current from power source 122 and control circuit 120 to reach roller-ball 170. Or in some embodiments, a layer of conductive material may provide a pathway from conductive post 128. For example, a bottom portion of reservoir 130 may be conductive and in conductive connection with an inside coating of conductive material in the interior portion of reservoir 132. Similarly, reservoir walls 132 may include one or more conductors extending to ring 172 that would allow an ionic fluid in reservoir 130 to be charged and conduct electricity to complete a circuit when iontophoretic roller-ball applicator 100 is being used.

As such, in some embodiments the fluid in reservoir 130 may function as the conductor. In such embodiments, roller-ball 170 may be formed of a non-conductive material, the current being conveyed by the fluid flowing from reservoir 130 to skin 5. Iontophoresis may cease when the fluid is depleted, by breaking the circuit and thereby automatically stopping the iontophoresis treatment.

Reservoir 130 may be replaceable or removable in some embodiments where iontophoretic roller-ball applicator 100 is reusable. In other embodiments, reservoir 130 may be affixed to the upper portion of body 110. Similarly, reservoir 130 may fit snugly in body 110 as a selectively removable attachment. As shown, fitment 160 may be coupled to reservoir 130 through cooperative connection of complimentary threads 134 of reservoir 130 and threads 164 of fitment 160.

In some embodiments where iontophoretic roller-ball applicator 100 is not reusable, fitment and reservoir 130 may be permanently affixed using adhesive or other suitable affixment. Similarly, fitment 160 may be permanently affixed to body 110 such that when reservoir 130 is empty, iontophoretic roller-ball applicator 100 may cease to function and would no longer be useful. In such embodiments and other reusable embodiments, no current would flow when iontophoretic roller-ball applicator 100 is in an upright position and no fluid is present outside of reservoir 130, or when the fluid level in reservoir 130 does not permit an electrical connection between roller-ball 170 and conductive post 128.

Ring 172 may be located in fitment 160 and on top of reservoir 130 to provide a seat for roller-ball 170. Ring 172 may be formed as part of fitment 160, or it may be compressed between reservoir 130 and fitment 160 when assembled. Ring 172 may be conductive and attach to a conductive portion of reservoir 130 to allow current to flow to roller-ball 170. In some embodiments, fitment 160 may be formed of a non conductive material having sufficient elasticity that roller-ball 170 may be press-fit into fitment 160 and retained by the top portion of fitment 160, as the top portion returns to its diameter. The diameter of the top portion of fitment 160 may be smaller than the diameter of roller-ball 170 to keep roller-ball 170 from falling out.

In other embodiments, fitment 160 may be formed of a conductive material, such as metal, that would not permit press-fitting of roller-ball 170. In such embodiments, roller-ball 170 may be placed inside of fitment 160 through the bottom prior to attachment with reservoir 130 and body 110, and held in place with ring 172. Roller-ball 170 may fit relatively loosely within fitment 160 to allow for free rotation and to convey ionic fluid from reservoir 130 to skin 5 when in use.

Roller-ball 170 may be formed of a conductive material that forms part of the electrical circuit, or it may be formed of a non-conductive material, allowing the current to pass through the fluid as described above. Cap 150 may connect to fitment through fitment threads 162 and cap threads 152 to reduce evaporation, leaking, and spillage of a fluid in reservoir 130. Cap 150 may be formed of any suitable material, such as metal, plastic, etc.

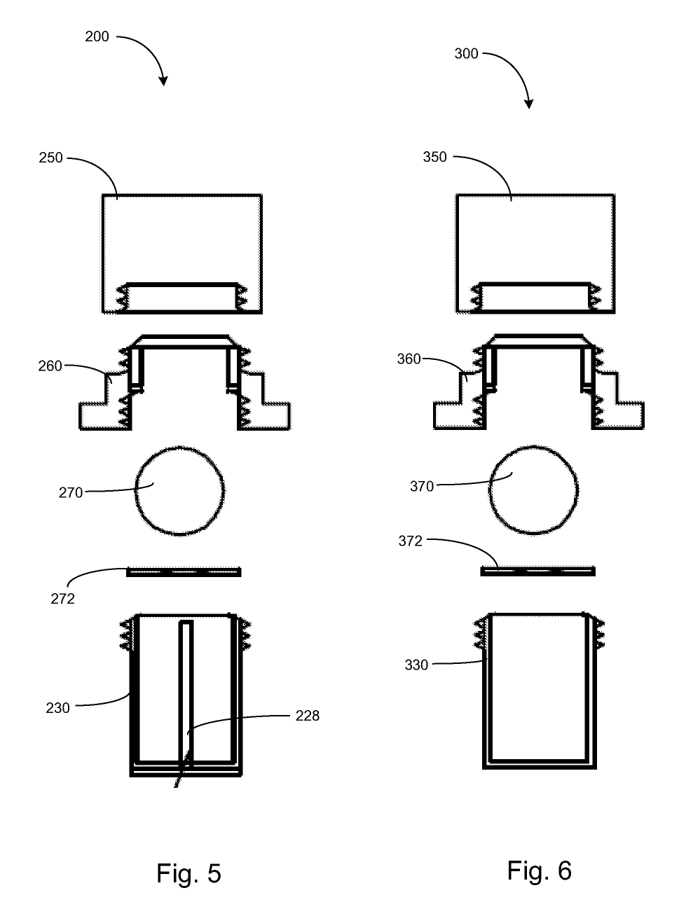

FIGS. 5-7 illustrate other embodiments of iontophoretic roller-ball applicators, particularly relating to mechanisms for completing a circuit for the iontophoresis. In each of these embodiments, corresponding components to iontophoretic roller-ball applicator 100 are similarly numbered. For example, roller-ball 270 of iontophoretic roller-ball applicator 200 corresponds to roller-ball 170 of iontophoretic roller-ball applicator 100.

In FIG. 5, iontophoretic roller-ball applicator 200 conductive post 228 extends into the center of reservoir 230 and up to roller-ball 270. Similar to iontophoretic roller-ball applicator 100, conductive post 228 may contact roller-ball 270 or may leave a gap between conductive post 228 and roller-ball 270 such that the fluid in reservoir 230 forms part of the circuit and that iontophoretic roller-ball applicator 200 stops functioning when the fluid is depleted. Ring 272, fitment 260, roller-ball 370, and cap 250 may be formed and function in the various embodiments as described above with respect to the similar components of iontophoretic roller-ball applicator 100.

In FIG. 6, iontophoretic roller-ball applicator 300 reservoir 330 may be formed of or coated with a conductive material such that ring 372 or fluid in reservoir 330 may provide electrical connection for the iontophoretic process when iontophoretic roller-ball applicator 300 is used. Ring 372, fitment 360, roller-ball 370, and cap 350 may be formed and function in the various embodiments as described above with respect to the similar components of iontophoretic roller-ball applicator 100.

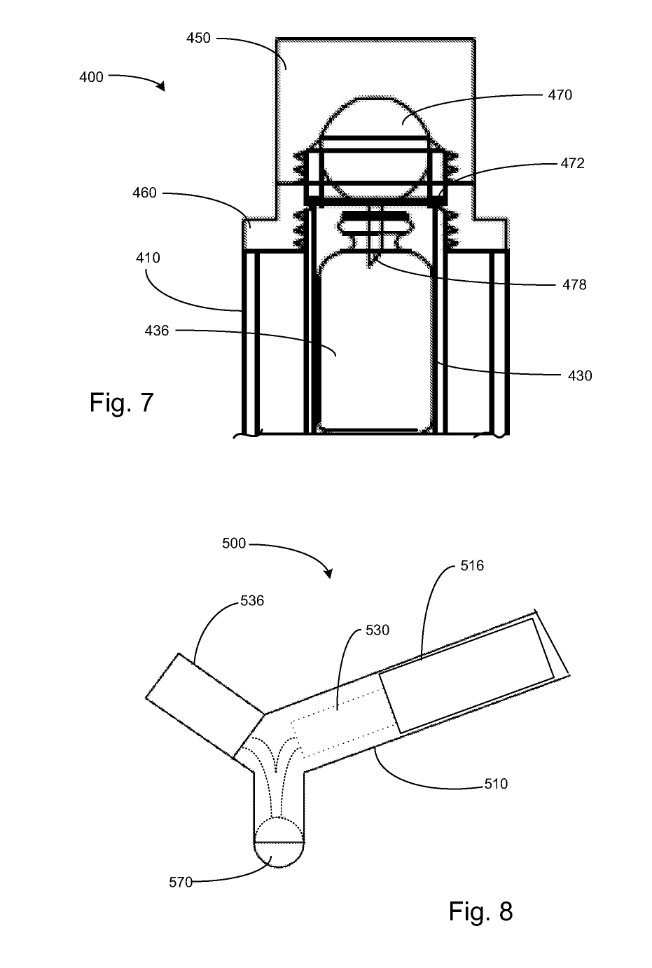

In FIG. 7, iontophoretic roller-ball applicator 400 may be reusable and provided to use a standard liquid medicament vial 436. Vial 436 may be positioned in body 410 in well 430. Well 430 may provide an electrical pathway to ring 472 from a power source, similar to the embodiments discussed above. Ring 472 may include needle 476 attached to ring 472 or formed in the ring. Needle 476 may be used to pierce the top of vial 430 similar to how hypodermic needles are used to pierce the same vials in other applications. Fitment 460, roller-ball 470, and cap 450 may be formed and function in the various embodiments as described above with respect to the similar components of iontophoretic roller-ball applicator 100.

Turning now to FIG. 8, iontophoretic roller-ball applicator 500 may include reservoir 536 along with reservoir 530 to provide ionic fluid to roller-ball 570 concurrently or sequentially, depending on the contents of the reservoirs and the desired treatment regimen. For example, some medications may be more effective when used with other medications, or with a skin conditioner. Similarly, other embodiments may provide three or more different reservoirs.

Additionally, body 510 may be shaped in a different ergonomic configuration than iontophoretic roller-ball applicator 100, as shown in other figures and described above. Similarly, electrode 516 may be contoured to fit a hand and body 510 may be angled to give a gentler wrist position of an individual holding the applicator. The various components of iontophoretic roller-ball applicator 500 may be formed and function similarly to those of the various embodiments as described above with respect to the similar components of iontophoretic roller-ball applicator 100. In some embodiments, a single reservoir 536 may be positioned as shown in FIG. 8 to allow for easy changing or refilling of reservoir 536.

In some embodiments, multiple roller-balls may be employed to engage a broader region. The bodies and handles of various iontophoretic roller-ball applicators may be formed and shaped to be of varying sizes and shapes depending on the desired aesthetic and ergonomic considerations of the applicator being used. Similarly, iontophoretic roller-ball applicators may be formed with specific shapes and sizes to accommodate particular areas of the body. For example, very small roller-balls may be used with iontophoretic roller-ball applicators for treating areas under the eyes, or fine wrinkles around the mouth and eyes, while a large ball may be used to treat large muscle groups such as those in legs, arms, and torso.

In other embodiments, iontophoretic roller-ball applicators may include a display in the body to indicate the amount of charge, time, and/or reservoir capacity used or remaining. Similarly, the iontophoretic roller-ball applicators may include switches to turn on the applicators and/or adjust the current to a desired level. Iontophoretic roller-ball applicators may turn on automatically with a circuit is completed between the electrodes and described above.

The various iontophoretic roller-ball applicators described may be used to provide several advantages over previous iontophoretic application systems. For example, medications or treatments may be made over a larger area of skin and a user may concentrate with varying intensities of particular trouble spots of on a region with an irregular shape that would be difficult to treat effectively with an iontophoretic patch.

Additionally, medications delivered by an active iontophoretic system may bypass the digestive system, which reduces digestive tract irritation. In many cases, ionic solutions administered orally may less potent than if administered transcutaneously. In compensation, it may be necessary in achieving a target effective dosage level to administer orally larger quantities of ionic solution than would be administered transcutaneously.

Active iontophoretic systems may not require intensive skin site sanitation to avoid infections. Equipment used in active iontophoresis may not interact with bodily fluids and, accordingly, need not be disposed as hazardous biological materials following use. Being a noninvasive procedure, the administration of ionic solution using an active iontophoretic system may not cause tissue injury of the types observed with hypodermic injections and with intravenous catheterizations. Repeated needle punctures in a single anatomical region, or long term catheter residence, can adversely affect the health of surrounding tissue. Needle punctures and catheter implantations inherently involve the experience of some degree of pain. These unintended consequences of invasive transcutaneous ionic solution administration are particularly undesirable in an area of the body that, being already injured, is to be treated directly for that injury with an ionic solution. Such might be the case, for example, in the treatment of a strained muscle or tendon.

The dosage of an ionic solution delivered iontophoretically may be conveniently and accurately measured by monitoring the amount and the duration of the current flowing during the administration. Similarly, the application may be completed with fluid contained in the reservoirs is depleted and the system stops. As such, the successful operation of an active iontophoretic system is not reliant on the medical skills of nurses or doctors. Foregoing the involvement of such medical personnel in the administration of ionic solutions whenever appropriate favors the convenience of patients and reduces the costs associated with the delivery of such types of therapy.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, examples are meant to be illustrative only and should not be construed to be limiting in any manner.

1. An iontophoretic device comprising:
a reservoir;
a roller-ball applicator coupled to the reservoir and configured to dispense contents of the reservoir;
a power supply;
a first electrode electrically coupled to a pole of the power supply; and
a second electrode coupled to the other pole of the power supply, wherein the device is configured such that a current travels between the first electrode and the second electrode through the skin of a subject when the device is used.

2. The device of claim 1, wherein the roller-ball applicator includes the first electrode.

3. The device of claim 2, wherein the roller ball applicator includes a roller-ball and a fitment, the roller-ball comprising a conductive material.

4. The device of claim 3, wherein the roller-ball functions as the first electrode.

5. The device of claim 1, further comprising a handle, wherein the second electrode is included on the handle.

6. The device of claim 1, wherein the reservoir contains an ionic solution.

7. The device of claim 1, further comprising a control circuit disposed between the power supply and one of the first and second electrodes.

8. The device of claim 1, wherein the device is configured for a single use only.

9. The device of claim 1, wherein the reservoir is refillable or replaceable.

10. The device of claim 1, further comprising a cap to fit over the roller-ball applicator.

11. The device of claim 1, the second electrode is configured to be grasped by the hand of the subject.

12. A method of iontophoretic application, the method comprising:
placing ionic fluid in a reservoir;
coupling the reservoir to a roller-ball applicator, the roller-ball applicator having a roller-ball and a handle;
grasping the handle;
placing the roller-ball against the skin of an individual;
dispensing the ionic fluid by way of the roller-ball onto the skin of the individual; and
applying current between the roller-ball and the skin of the individual such that the ionic fluid is delivered iontophoretically into the skin of the individual.

13. The method of claim 12, further comprising applying the current until the ionic fluid is substantially depleted from the reservoir.

14. The method of claim 12, further comprising applying the current for a pre-determined amount of time.

15. The method of claim 12, wherein the current is supplied by a power supply electrically coupled to the reservoir and the handle, wherein the person grasping the handle is the individual, and wherein current is conducted through the handle to the hand of the individual.

16. The method of claim 12, wherein the roller-ball is formed from a conductive material.

17. The method of claim 12, wherein the roller-ball is formed of a non-conductive material.

18. The method of claim 12, wherein a control circuit is located in the handle and controls the application of current.

19. The method of claim 12, wherein the reservoir is configured for a single use only.

20. The method of claim 1, wherein the reservoir is refillable or replaceable.
